(19) United States
(12) Patent Application Publication
(10) Pub. No.: US 2023/0041510 A1
(43) Pub. Date: Feb. 9, 2023

(54) REMOTE DIAGNOSIS AND TREATMENT SYSTEM AND REMOTE DIAGNOSIS AND TREATMENT METHOD

(71) Applicants: Acer Incorporated, New Taipei City (TW); Acer Medical Inc., New Taipei City (TW)

(72) Inventors: Chun-Lin Han, New Taipei City (TW); Yin-Hsong Hsu, New Taipei City (TW); Shu-Jun Liao, New Taipei City (TW); Yung-Han Chang, New Taipei City (TW); Ming-Chun Yu, New Taipei City (TW)

(73) Assignees: Acer Incorporated, New Taipei City (TW); Acer Medical Inc., NEW TAIPEI CITY (TW)

(21) Appl. No.: 17/697,952

(22) Filed: Mar. 18, 2022

(30) Foreign Application Priority Data
Aug. 9, 2021 (TW) 110129329

(51) Int. Cl. G16H 40/20; H04L 65/1093

(52) U.S. Cl. CPC G16H 40/20; H04L 65/1093

(57) ABSTRACT

A remote diagnosis and treatment system and a remote diagnosis and treatment method are provided. The remote diagnosis and treatment method includes: establishing an online clinic room and adding a first terminal device and a second terminal device to the online clinic room by a server; in response to receiving a first end signal from the first terminal device, removing the first terminal device from the online clinic room, adding a patient corresponding to the second terminal device to a service-waiting queue, and transmitting the service-waiting queue to a third terminal device by the server; transmitting a first join signal corresponding to the second terminal device to the server according to the service-waiting queue by the third terminal device; and adding the third terminal device to the online clinic room according to the first join signal by the server.

### CROSS-REFERENCE TO RELATED APPLICATION

This application claims the priority benefit of Taiwan application serial no. 110129329, filed on Aug. 9, 2021. The entirety of the above-mentioned patent application is hereby incorporated by reference herein and made a part of this specification.

### BACKGROUND

### Technical Field

The disclosure relates to a remote diagnosis and treatment system and a remote diagnosis and treatment method.

### Description of Related Art

In recent years, many doctors and patients have begun to conduct medical consultations via video. Before a video consultation begins, the patient must wait for the doctor to make a video call at an agreed time. During the video consultation, the doctor may need to explain some things that are not necessary to be explained by the doctor (such educational health instructions for the patient), which may waste a great deal of time. If the doctor chooses to transfer the patient to the nursing staff for the nursing staff to explain his/her instructions, the nursing staff will need to make a new video call to the patient.

On the other hand, with video consultation, the nursing staff has no way of knowing whether or not the patient is ready for consultation or ready to receive doctor's instructions, and can only passively wait for notification from the doctor. In addition, in the handover process in which the patient is transferred to the nursing staff, the patient has no way of knowing whether or when the nursing staff will call him. It is also difficult for the hospital to track whether or not nursing staff have completed their assigned tasks instructed by the doctor.

### SUMMARY

The disclosure provides a remote diagnosis and treatment system and a remote diagnosis and treatment method, which can assist a hospital to manage an online diagnosis and treatment process of a patient.

A remote diagnosis and treatment system of the disclosure includes a server, a first terminal device, a second terminal device, and a third terminal device. The first terminal device is communicatively connected to the server. The second terminal device is communicatively connected to the server. The third terminal device is communicatively connected to the server. The server establishes an online clinic room and adds a first terminal device and a second terminal device to the online clinic room. In response to receiving a first end signal from the first terminal device, the server removes the first terminal device from the online clinic room, adds a patient corresponding to the second terminal device to a service-waiting queue, and transmits the service-waiting queue to the third terminal device. The third terminal device transmits a first join signal corresponding to the second terminal device to the server according to the service-waiting queue. The server adds the third terminal device to the online clinic room according to the first join signal.

In an embodiment of the disclosure, the first end signal includes information on assigned tasks, in which the server adds the patient to the service-waiting queue in response to receiving the information on assigned tasks.

In an embodiment of the disclosure, the server removes the patient from the service-waiting queue in response to receiving the first join signal.

In an embodiment of the disclosure, in response to receiving a second end signal including an updated information on assigned tasks from the third terminal device, the server removes the third terminal device from the online clinic room and adds the patient back to the service-waiting queue so as to generate an updated service-waiting queue.

In an embodiment of the disclosure, the remote diagnosis and treatment system further includes a fourth terminal device. The fourth terminal device is communicatively connected to the server. The server transmits the updated service-waiting queue to the fourth terminal device. The fourth terminal device transmits a second join signal corresponding to the second terminal device to the server according to the updated service-waiting queue. The server adds the fourth terminal device to the online clinic room according to the second join signal.

In an embodiment of the disclosure, the server closes the online clinic room in response to receiving a second end signal from the second terminal device and receiving a third end signal from the third terminal device.

In an embodiment of the disclosure, the server transmits a notification signal to the second terminal device in response to receiving the information on assigned tasks, in which the notification signal includes the service-waiting queue.

In an embodiment of the disclosure, the third terminal device receives a user operation so as to select the patient from a plurality of patient in the service-waiting queue, and transmits the first join signal corresponding to the patient to the server.

In an embodiment of the disclosure, the first terminal device receives a treatment-waiting queue from the server, in which the treatment-waiting queue includes the patient. The first terminal device transmits a number-calling signal corresponding to the patient to the server according to the treatment-waiting queue. The server transmits a dialing signal to the second terminal device according to the number-calling signal. The server adds the second terminal device to the online clinic room in response to receiving a first reply signal corresponding to the dialing signal from the second terminal device.

In an embodiment of the disclosure, the server removes the patient from the treatment-waiting queue in response to receiving the number-calling signal. The server adds the patient back to the treatment-waiting queue in response to receiving a second reply signal corresponding to the dialing signal from the second terminal device.

In an embodiment of the disclosure, the server removes the patient from the treatment-waiting queue in response to receiving the number-calling signal. The second terminal device transmits a third reply signal corresponding to the dialing signal to the server in response to not receiving a user operation after a preset period of time after receiving the dialing signal. The server adds the patient back to the treatment-waiting queue in response to receiving the third reply signal from the second terminal device.

In an embodiment of the disclosure, the first terminal device receives a user operation so as to select the patient from a plurality of patient in the treatment-waiting queue, and transmits the number-calling signal corresponding to the patient to the server.

In an embodiment of the disclosure, the second terminal device outputs a prompt message in response to receiving the dialing signal.

In an embodiment of the disclosure, the second terminal device receives appointment information from the server, in which the appointment information includes a check-in period. The server adds the patient corresponding to the second terminal device to a treatment-waiting queue in response to receiving a check-in signal from the second terminal device during the check-in period, and transmits the treatment-waiting queue to the second terminal device.

In an embodiment of the disclosure, the server transmits a notification signal to the second terminal device in response to not receiving the check-in signal during the preset period.

A remote diagnosis and treatment method of the disclosure includes the following. An online clinic room is established and a first terminal device and a second terminal device are added to the online clinic room by a server. In response to receiving a first end signal from the first terminal device, the first terminal device is removed from the online clinic room, a patient corresponding to the second terminal device is added to a service-waiting queue, and the service-waiting queue is transmitted to a third terminal device by the server. A first join signal corresponding to the second terminal device is transmitted to the server according to the service-waiting queue by the third terminal device. The third terminal device is added to the online clinic room according to the first join signal by the server.

Based on the above, the disclosure provides a complete diagnosis and treatment for doctors, nursing staff, or patients. The disclosure also assists the doctor in transferring the patient to one or more nursing staff such that the one or more nursing staff can give educational health instructions to the patient, thereby reducing the cost of time in transfer of the patient.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a schematic diagram of a remote diagnosis and treatment system according to an embodiment of the disclosure.

FIG. 2 is a schematic diagram illustrating a check-in procedure according to an embodiment of the disclosure.

FIG. 3 is a schematic diagram illustrating a check-in procedure according to another embodiment of the disclosure.

FIG. 4 is a schematic diagram illustrating a number-calling procedure according to an embodiment of the disclosure.

FIG. 5 is a schematic diagram illustrating a number-calling procedure according to another embodiment of the disclosure.

FIG. 6 is a schematic diagram illustrating a waiting procedure after a diagnosis and treatment is completed according to an embodiment of the disclosure.

FIG. 7 is a schematic diagram illustrating a waiting procedure after a diagnosis and treatment is completed according to another embodiment of the disclosure.

FIG. 8 is a schematic diagram illustrating a procedure for performing assigned tasks from a doctor according to an embodiment of the disclosure.

FIG. 9 is a schematic diagram illustrating a procedure for performing assigned tasks from a doctor according to another embodiment of the disclosure.

FIG. 10 is a schematic diagram illustrating a procedure for performing assigned tasks from a doctor according to yet another embodiment of the disclosure.

FIG. 11 is a schematic diagram illustrating a procedure for performing assigned tasks from a doctor according to still another embodiment of the disclosure.

FIG. 12 is a flowchart illustrating a remote diagnosis and treatment method according to an embodiment of the disclosure.

### DESCRIPTION OF THE EMBODIMENTS

To make the content of the disclosure more easily understood, the following specific embodiments are given as examples according to which the disclosure can indeed be implemented. Wherever possible, the same reference numbers are used in the drawings and the description to refer to the same or like parts/components/steps.

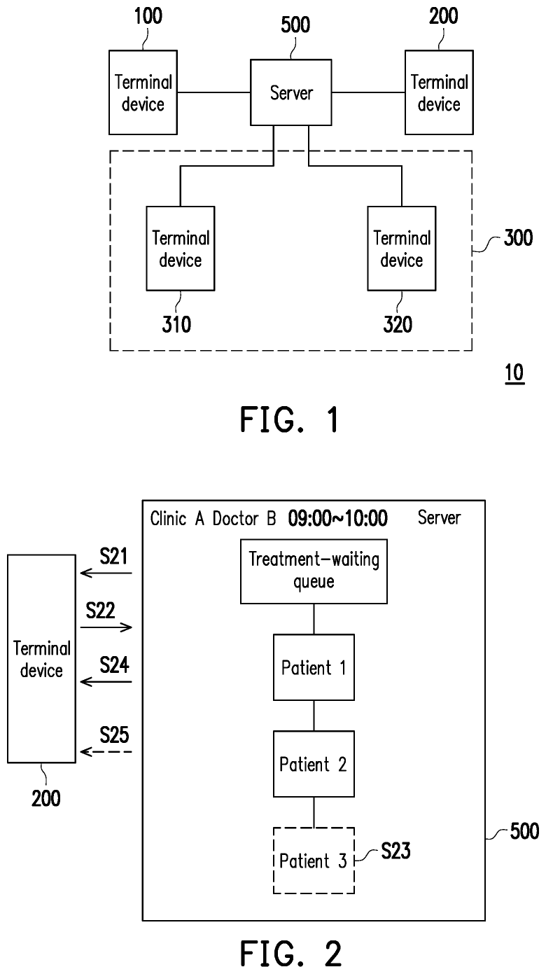

FIG. 1 is a schematic diagram of a remote diagnosis and treatment system 10 according to an embodiment of the disclosure. The remote diagnosis and treatment system 10 may include a server 500, a terminal device 100, a terminal device 200, and at least one terminal device 300. The terminal device 100 may be held by a doctor, the terminal device 200 may be held by a patient (or a person waiting for clinic), and the at least one terminal device 300 may be held by at least one nursing staff member. The server 500 may be communicatively connected to the terminal device 100, the terminal device 200, or at least one terminal device 300. The at least one terminal device 300 may include N terminal devices, where N is any positive integer. In this embodiment, it is assumed that N is equal to 2. Therefore, the at least one terminal device 300 may include two terminal devices, such as a terminal device 310 and a terminal device 320. The terminal device 310 and the terminal device 320 may be held by different nursing staff members, respectively.

The server 500 (or the terminal device 100, the terminal device 200, the terminal device 310, or the terminal device 320) may include a server computer, a desktop computer, a notebook computer, a network computer, a workstation, a personal digital assistant (PDA), a personal computer (PC), a tablet computer, a telephone device, and other electronic devices with computing and communication capabilities. The server 500 (or the terminal device 100, the terminal device 200, the terminal device 310, or the terminal device 320) may include, but is not limited to, necessary elements to run the server 500 (or the terminal device 100, the terminal device 200, the terminal device 310, or the terminal device 320), such a display, a touch-sensitive display, a speaker, an input-output device, a transceiver circuit, an analog-to-digital (A/D) converter, a digital-to-analog (D/A) converter, a processing circuit, a memory circuit, or one or more antenna units, etc. The server 500 (or the terminal device 100, terminal device 200, terminal device 310, or terminal device 320) may interact with an operator through a user interface (UI). The user interface may include applications such as a browser.

FIG. 2 is a schematic diagram illustrating a check-in procedure according to an embodiment of the disclosure. A patient may join a treatment-waiting queue according to a check-in procedure. In this embodiment, it is assumed that the server 500 has stored the patient's appointment information in advance, wherein the appointment information may include a check-in period. The patient may be allowed to check in during the check-in period. The server 500 may store the patient's appointment information after the patient is registered.

In step S21, the terminal device 200 may receive appointment information from the server 500. The terminal device 200 may display the appointment information including the check-in period and other information through a user interface for the patient's reference. The patient may refer to the appointment information and decide when to check in.

In step S22, the terminal device 200 may transmit a check-in signal to the server 500 during the check-in period. To be specific, the terminal device 200 may display a button for check-in through the user interface. When the patient decides to check in, he or she can operate the terminal device 200 by pressing the button to transmit the check-in signal to the server 500.

In step S23, the server 500 may add the patient corresponding to the terminal device 200 to the treatment-waiting queue in response to receiving the check-in signal from the terminal device 200 during the check-in period. In this embodiment, it is assumed that the owner of the terminal device 200 is “patient 3”. The server 500 may add the “patient 3” to the treatment-waiting queue. The patient newly added to the treatment-waiting queue may be placed at the end of the treatment-waiting queue and have a lower priority. In the treatment-waiting queue, the patient with higher priority may have his or her waiting number called out earlier by the doctor to start diagnosis and treatment.

The server 500 may store the treatment-waiting queue. The terminal device 100, the terminal device 200, the terminal device 310, or the terminal device 320 may access the server 500 to obtain the treatment-waiting queue.

In step S24, the server 500 may transmit the treatment-waiting queue to the terminal device 200. The terminal device 200 may display the information relevant to the treatment-waiting queue through the user interface for the patient's reference. For example, the patient may get to know from the treatment-waiting queue his or her own position in the treatment-waiting queue. The patient may judge the waiting time for diagnosis and treatment according to his or her own position in the treatment-waiting queue.

In step S25, the server 500 may transmit the updated treatment-waiting queue to the terminal device 200. In one embodiment, the server 500 may transmit the updated treatment-waiting queue to the terminal device 200 in response to the treatment-waiting queue being updated. In another embodiment, the server 500 may periodically transmit the updated treatment-waiting queue to the terminal device 200.

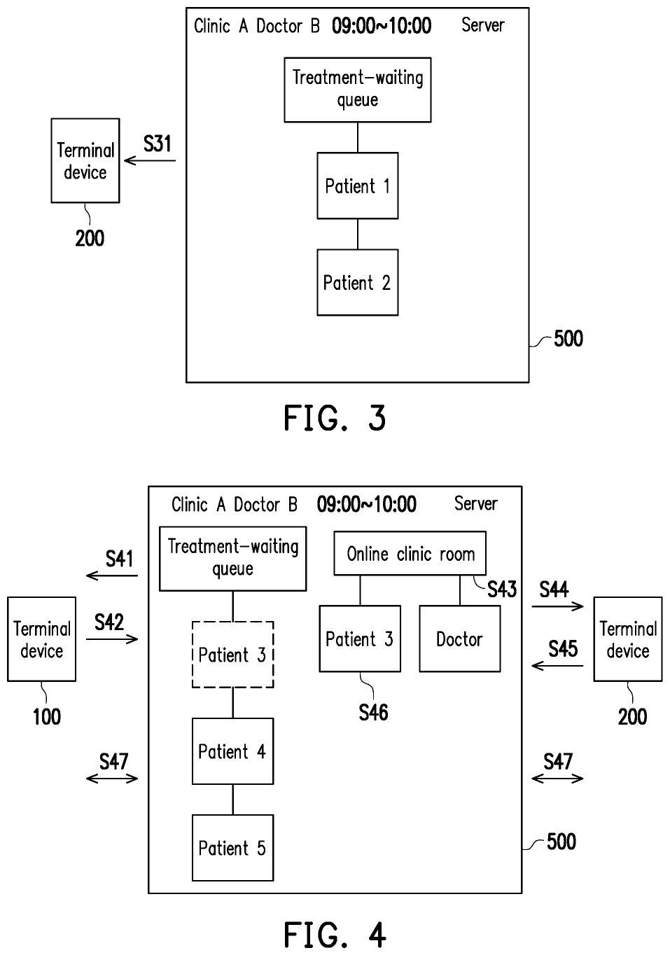

FIG. 3 is a schematic diagram illustrating a check-in procedure according to another embodiment of the disclosure. In step S31, the server 500 may judge that a patient is unable to check in in response to not receiving a check-in signal from the terminal device 200 during the check-in period. Accordingly, the server 500 may transmit a notification signal to the terminal device 200, so as to remind the patient to make an appointment again.

FIG. 4 is a schematic diagram illustrating a number-calling procedure according to an embodiment of the disclosure. FIG. 4 shows a situation in which a patient agrees to start diagnosis and treatment after his or her waiting number is called out by the doctor. In step S41, the terminal device 100 may receive a treatment-waiting queue from the server 500, wherein the treatment-waiting queue may include the patient holding the terminal device 200. The terminal device 100 may display the treatment-waiting queue through the user interface for the doctor to select a patient.

In step S42, the terminal device 100 may transmit a number-calling signal of the patient corresponding to the terminal device 200 to the server 500 according to the treatment-waiting queue. To be specific, after referring to the treatment-waiting queue, if the doctor decides to start online diagnosis and treatment for the patient holding the terminal device 200 (i.e. the patient 3), the doctor may operate the terminal device 100. The terminal device 100 may receive the doctor's user operation through the user interface so as to select the “patient 3” from a plurality of patients in the treatment-waiting queue, and transmit the number-calling signal corresponding to the “patient 3” to the server 500.

In step S43, the server 500 may establish an online clinic room according to the number-calling signal, and may add the terminal device 100 to the online clinic room. In one embodiment, the server 500 may remove the patient holding the terminal device 200 from the treatment-waiting queue after receiving the number-calling signal corresponding to the terminal device 200.

In step S44, the server 500 may transmit a dialing signal to the terminal device 200 according to the number-calling signal. After receiving the dialing signal, the terminal device 200 may output a prompt message to remind the patient to start the diagnosis and treatment. The prompt message may be an audio message or a video message.

In step S45, the terminal device 200 may transmit a first reply signal to the server 500, wherein the first reply signal may indicate that the patient agrees to start the diagnosis and treatment. To be specific, if the patient agrees to start the diagnosis and treatment, the patient may operate the terminal device 200. The terminal device 200 may receive the operation of the patient through the user interface so as to transmit the first reply signal to the server 500.

In step S46, the server 500 may add the terminal device 200 to the online clinic room in response to receiving the first reply signal corresponding to the dialing signal from the terminal device 200. In one embodiment, the server 500 may remove the patient holding the terminal device 200 from the treatment-waiting queue in response to adding the terminal device 200 to the online clinic room.

In step S47, the server 500 may arrange for the terminal device 100 and the terminal device 200 to communicate with each other. For example, the server 500 may establish a network connection or a multimedia session for members of the online clinic room (e.g. the terminal device 100 and the terminal device 200). The members of the online clinic room may communicate with each other through the network connection or the multimedia session. The doctor may communicate with the patient through the online clinic room and conduct diagnosis and treatment for the patient.

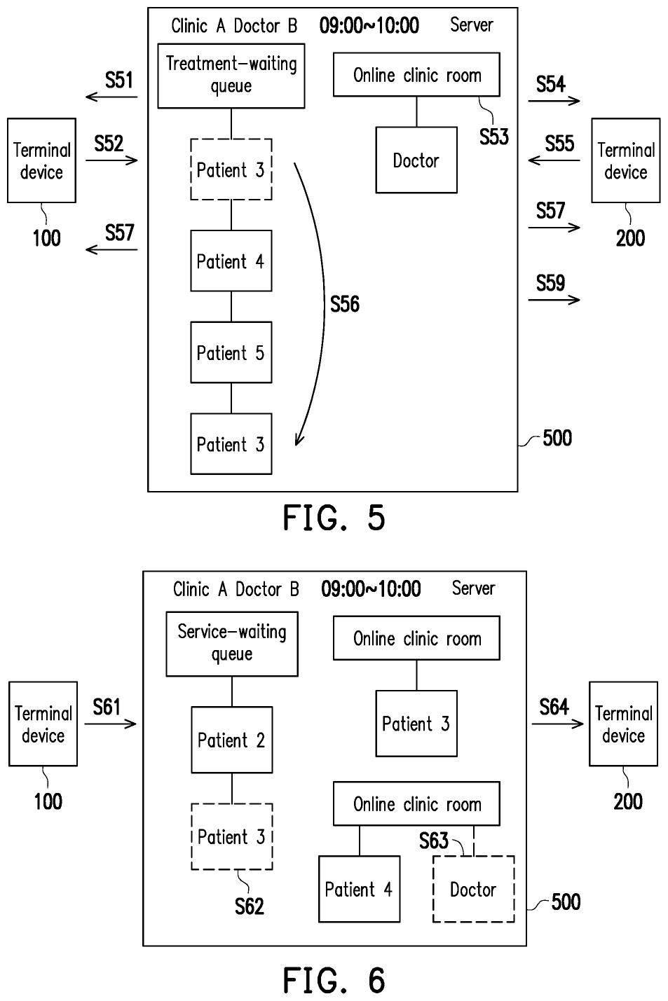

FIG. 5 is a schematic diagram illustrating a number-calling procedure according to another embodiment of the disclosure. FIG. 5 shows a situation in which a patient refuses to start diagnosis and treatment or the patient does not respond to the number-calling after the doctor calls out the patient's waiting number. In step S51, the terminal device 100 may receive a treatment-waiting queue from the server 500, wherein the treatment-waiting queue may include the patient holding the terminal device 200. The terminal device 100 may display the treatment-waiting queue through the user interface for the doctor to select a patient.

In step S52, the terminal device 100 may transmit a number-calling signal of the patient corresponding to the terminal device 200 to the server 500 according to the treatment-waiting queue. To be specific, after referring to the treatment-waiting queue, if the doctor decides to start online diagnosis and treatment for the patient holding the terminal device 200 (i.e. the patient 3), the doctor may operate the terminal device 100. The terminal device 100 may receive the doctor's user operation through the user interface so as to select the “patient 3” from a plurality of patients in the treatment-waiting queue, and transmit the number-calling signal corresponding to the “patient 3” to the server 500.

In step S53, the server 500 may establish an online clinic room according to the number-calling signal, and may add the terminal device 100 to the online clinic room. In one embodiment, the server 500 may remove the patient holding the terminal device 200 from the treatment-waiting queue after receiving the number-calling signal corresponding to the terminal device 200.

In step S54, the server 500 may transmit a dialing signal to the terminal device 200 according to the number-calling signal. After receiving the dialing signal, the terminal device 200 may output a prompt message to remind the patient to start the diagnosis and treatment. The prompt message may be an audio message or a video message.

In step S55, the terminal device 200 may transmit a second reply signal or a third reply signal to the server 500, the second reply signal may indicate that the patient refuses to start the diagnosis and treatment, and the third reply signal may indicate that the patient does not agree or that the patient refuse to start the diagnosis and treatment; that is, the patient does not respond to the dialing signal.

To be specific, if the patient refuses to start the diagnosis and treatment, the patient may operate the terminal device 200. The terminal device 200 may receive the patient's user operation through the user interface so as to transmit the second reply signal corresponding to the dialing signal to the server 500. On the other hand, if the patient does not respond to the dialing signal, the terminal device 200 may transmit the third reply signal corresponding to the dialing signal to server 500 in response to not receiving the patient's user operation during a preset period after receiving the dialing signal.

In step S56, the server 500 may add the patient holding the terminal device 200 (i.e. the patient 3) back to the treatment-waiting queue in response to receiving the second reply signal or the third reply signal from the terminal device 200. To be specific, assuming that the “patient 3” is removed from the treatment-waiting queue by the server 500 in step S53, after receiving the second reply signal or the third reply signal from the terminal device 200, the server 500 may judge that the online diagnosis and treatment of the “patient 3” has not been completed. Accordingly, the server 500 may judge that the “patient 3” has missed the number-calling of his or her number, and may add the “patient 3” back to the treatment-waiting queue. After the “patient 3” is added back the treatment-waiting queue, the priority of him or her in the treatment-waiting queue may be lowered by the server 500. On the other hand, assuming that the “patient 3” is not removed from the treatment-waiting queue by the server 500 in step S53, after receiving the second reply signal or the third reply signal from the terminal device 200, the server 500 may judge that the online diagnosis and treatment of the “patient 3” has not been completed. Accordingly, the server 500 may update the position of the “patient 3” in the treatment-waiting queue, so as to lower the priority of the patient in the treatment-waiting queue.

For example, server 500 may move “patient number 3” to the end of the treatment-waiting queue.

In step S57, the server 500 may transmit a notification signal to the terminal device 100 according to the reply signal received from the terminal device 200. If the reply signal is the second reply signal, the notification signal may indicate that the patient refuses the diagnosis and treatment. If the reply signal is the third reply signal, the notification signal may indicate that the patient has not responded to the doctor's number-calling. The notification signal may also include an updated treatment-waiting queue. The terminal device 100 may display the updated treatment-waiting queue through the user interface for the doctor's reference.

On the other hand, the server 500 may transmit the notification signal to the terminal device 200 according to the reply signal. The notification signal may indicate that the patient has missed the number-calling for his or her number. The notification signal may also include the updated treatment-waiting queue. The terminal device 200 may display the updated treatment-waiting queue through the user interface for the patient's reference.

In step S58, the server 500 may close the online clinic room corresponding to the terminal device 100 and the terminal device 200. After the online clinic room is closed, the network connection or multimedia session between the terminal device 100 and the terminal device 200 may be terminated. The doctor may operate the terminal device 100 to perform step S41 again to select a next patient.

In step S59, the server 500 may transmit the updated treatment-waiting queue to the terminal device 200. In one embodiment, the server 500 may transmit the updated treatment-waiting queue to the terminal device 200 in response to the treatment-waiting queue being updated. In another embodiment, the server 500 may periodically transmit the updated treatment-waiting queue to the terminal device 200.

FIG. 6 is a schematic diagram illustrating a waiting procedure after a diagnosis and treatment is completed according to an embodiment of the disclosure. After the completion of the diagnosis and treatment, if the doctor assigns tasks for the nursing staff, a patient will keep the terminal device 200 in the online clinic room and wait for the nursing staff to perform the tasks assigned by the doctor.

In step S61, the terminal device 100 may transmit an end signal to the server 500, wherein the end signal may include information on assigned tasks from the doctor. The end signal may also indicate that patient-related tasks are still pending. To be specific, after the doctor completes the diagnosis and treatment with the patient, the doctor may assign one or more tasks to at least one nursing staff member, such that the at least one nursing staff member may perform the assigned tasks for the patient. The doctor may operate the terminal device 100 to transmit the end signal including the information on assigned tasks to the server 500. The information on assigned tasks may contain information such as one or more pending tasks, the clinic number, or patient information.

In step S62, the server 500 may add the patient holding the terminal device 200 to a service-waiting queue in response to receiving the end signal containing the information on assigned tasks from the terminal device 100. The service-waiting queue may include one or more patients waiting to meet with the nursing staff. The service-waiting queue may also contain the information on assigned tasks. The terminal device may access the service-waiting queue stored in the server 500 to obtain the information on assigned tasks.

In step S63, the server 500 may remove the terminal device 100 from the online clinic room in response to receiving the end signal from the terminal device 100.

In step S64, the server 500 may transmit a notification signal to the terminal device 200, so as to remind the patient to keep the terminal device 200 in the online clinic room and wait for contact from the nursing staff. The notification signal may include information such as service-waiting queue or the information on assigned tasks. The terminal device 200 may output the information carried by the notification signal through the user interface for the patient's reference.

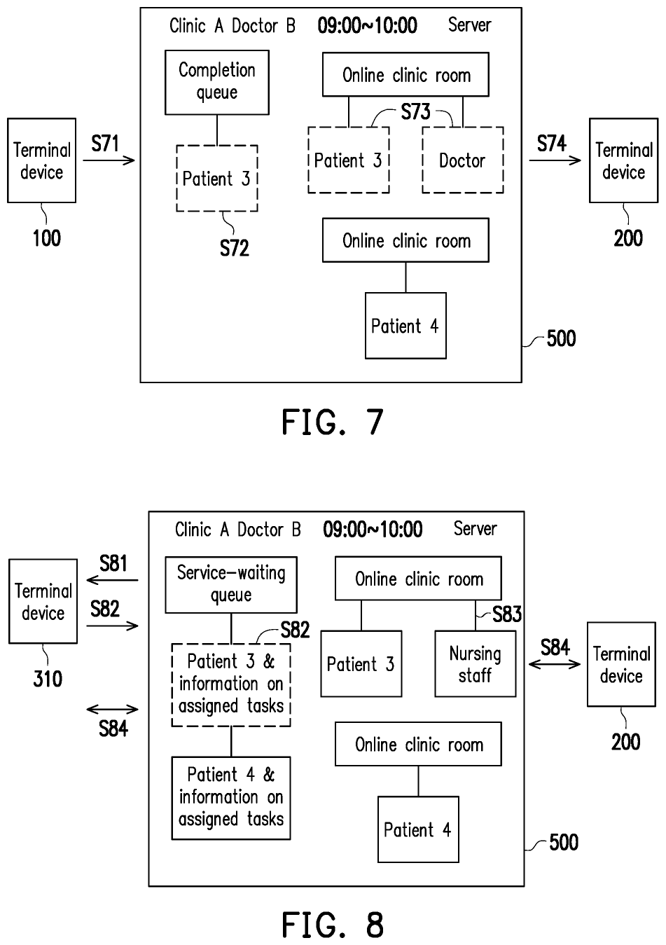

FIG. 7 is a schematic diagram illustrating a waiting procedure after a diagnosis and treatment is completed according to another embodiment of the disclosure. After the completion of the diagnosis and treatment, if the doctor does not assign any tasks, the terminal device 200 may leave the online clinic room to end the diagnosis and treatment process.

In step S71, the terminal device 100 may transmit an end signal to the server 500. The server 500 may judge that there is no pending tasks for a patient according to the end signal. In one embodiment, the server 500 may judge that there is no assigned tasks in response to the end signal not including information on assigned tasks. In another embodiment, the information contained in the end signal may indicate to the server 500 that there is no pending tasks for the patient.

In step S72, the server 500 may add the patient holding the terminal device 200 to the completion queue in response to receiving the end signal from the terminal device 100. The completion queue may include one or more patients who have completed the diagnosis and treatment process. The completion queue may also contain information such as the clinic number, or patient information. The terminal device accesses the completion queue stored in the server 500 to obtain the above-mentioned information.

In step S73, the server 500 may remove the terminal device 100 and the terminal device 200 from the online clinic room in response to receiving the end signal from the terminal device 100. In one embodiment, the server 500 may respectively remove the terminal device 100 and the terminal device 200 from the online clinic room in response to receiving the end signals from the terminal device 100 and the terminal device 200 respectively. After the terminal device 100 and the terminal device 200 are removed from the online clinic room, the server 500 may close the online clinic room.

In step S74, the server 500 may transmit a notification signal to the terminal device 200, so as to remind the patient that the diagnosis and treatment process is completed.

FIG. 8 is a schematic diagram illustrating a procedure for performing assigned tasks from a doctor according to an embodiment of the disclosure. After the completion of the diagnosis and treatment, the doctor may assign tasks to be performed by the nursing staff and transfer a patient to the nursing staff.

In step S81, the server 500 may transmit a service-waiting queue to the terminal device 310. The service-waiting queue may include information on assigned tasks (for example: completed tasks or pending tasks), the clinic number, or patient information, and other tasks. The terminal device 310 may display the service-waiting queue through the user interface for reference by the nursing staff holding the terminal device 310. The nursing staff may judge which patient the nursing staff needs to serve according to the service-waiting queue. In this embodiment, it is assumed that the patient to be served by the nursing staff is the owner of the terminal device 200 (i.e. the patient 3).

In step S82, the terminal device 310 may transmit a join signal corresponding to the terminal device 200 to the server 500 according to the service-waiting queue. To be specific, after referring to the service-waiting queue, if the nursing staff decides to provide services (e.g. educational health instruction) for the patient holding the terminal device 200 (i.e. the patient 3), the nursing staff may operate the terminal device 310. The terminal device 310 may receive the nursing staff's user operation through the user interface so as to select the “patient 3” from a plurality of patients in the service-waiting queue, and transmit a join signal corresponding to the “patient 3” to the server 500. In one embodiment, the server 500 may remove the patient holding the terminal device 200 from the service-waiting queue after receiving the join signal corresponding to the terminal device 200.

In step S83, the server 500 may add the terminal device 310 to the online clinic room corresponding to the terminal device 200 according to the join signal.

In step S84, the server 500 may arrange for the terminal device 310 and the terminal device 200 to communicate with each other. For example, the server 500 may establish a network connection or a multimedia session for members of the online clinic room (e.g. the terminal device 310 and the terminal device 200). The members of the online clinic room may communicate with each other through the network connection or the multimedia session. The nursing staff may communicate with the patient through the online clinic room and perform the assigned tasks for the patient.

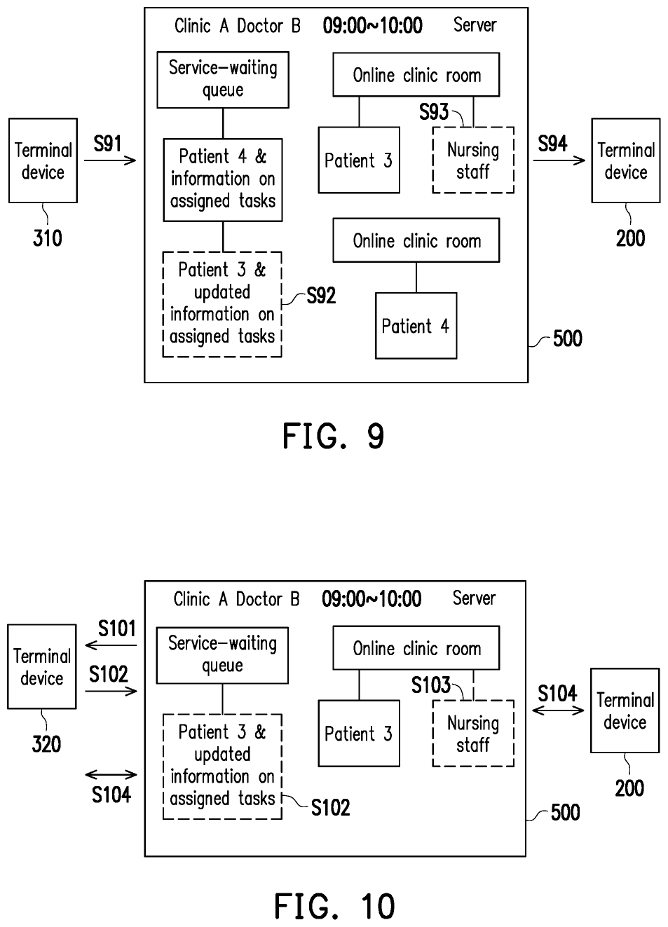

FIG. 9 is a schematic diagram illustrating a procedure for performing assigned tasks from a doctor according to another embodiment of the disclosure. The nursing staff may leave the online clinic room after completing all or part of the assigned tasks.

In step S91, the terminal device 310 may transmit an end signal to the server 500, wherein the end signal may include updated information on assigned tasks. The updated information on assigned tasks may contain completed tasks or pending tasks. For example, information on assigned tasks may include a plurality of pending tasks. When a nursing staff member holding the terminal device 310 completes a pending task assigned to him or her, the nursing staff may operate the terminal device 310 to update the completed pending task to a completed task, thereby generating an updated information on assigned tasks.

In step S92, the server 500 may add the patient holding the terminal device 200 (i.e. the patient 3) back to the service-waiting queue in response to receiving the end signal including the updated information on assigned tasks so as to generate an updated service-waiting queue. The patient newly added to the service-waiting queue may be placed at the end of the service-waiting queue and have a lower priority. In the service-waiting queue, the patient with higher priority may be served earlier by nursing staff.

In step S93, the server 500 may remove the terminal device 310 from the online clinic room in response to receiving the end signal from the terminal device 310.

In step S94, the server 500 may transmit a notification signal to the terminal device 200, so as to remind the patient to keep the terminal device 200 in the online clinic room and wait for contact from another nursing staff member. The notification signal may further contain the updated service-waiting queue. The terminal device 200 may display the updated service-waiting queue through the user interface for the patient's reference.

FIG. 10 is a schematic diagram illustrating a procedure for performing assigned tasks from a doctor according to yet another embodiment of the disclosure. After the nursing staff holding the terminal device 310 completes some of the assigned tasks, remaining pending tasks may be taken over by another nursing staff member holding the terminal device 320.

In step S101, the server 500 may transmit the updated service-waiting queue to the terminal device 320. The updated service-waiting queue may contain updated information on assigned tasks such as tasks (for example: completed tasks or pending tasks), the clinic number, or patient information. The terminal device 320 may display the updated service-waiting queue through the user interface for reference by the nursing staff holding the terminal device 320. The nursing staff may judge which patient the nursing staff needs to serve according to the updated service-waiting queue. In this embodiment, it is assumed that the patient to be served by the nursing staff is the owner of the terminal device 200 (i.e. the patient 3).

In step S102, the terminal device 320 may transmit a join signal corresponding to the terminal device 200 to the server 500 according to the updated service-waiting queue. To be specific, after referring to the updated service-waiting queue, if the nursing staff decides to provide services (e.g. educational health instructions) for the patient (i.e. the patient 3) holding the terminal device 200, the nursing staff may operate the terminal device 320. The terminal device 320 may receive the nursing staff's user operation through the user interface so as to select the “patient 3” from a plurality of patients in the updated service-waiting queue, and transmit a join signal corresponding to the “patient 3” to the server 500. In one embodiment, the server 500 may remove the patient holding the terminal device 200 from the updated service-waiting queue after receiving the join signal corresponding to the terminal device 200.

In step S103, the server 500 may add the terminal device 320 to the online clinic room corresponding to the terminal device 200 according to the join signal.

In step S104, the server 500 may arrange for the terminal device 320 and the terminal device 200 to communicate with each other. For example, the server 500 may establish a network connection or a multimedia session for members of the online clinic room (e.g. the terminal device 320 and the terminal device 200). The members of the online clinic room may communicate with each other through the network connection or the multimedia session. The nursing staff may communicate with the patient through the online clinic room and perform the assigned tasks for the patient.

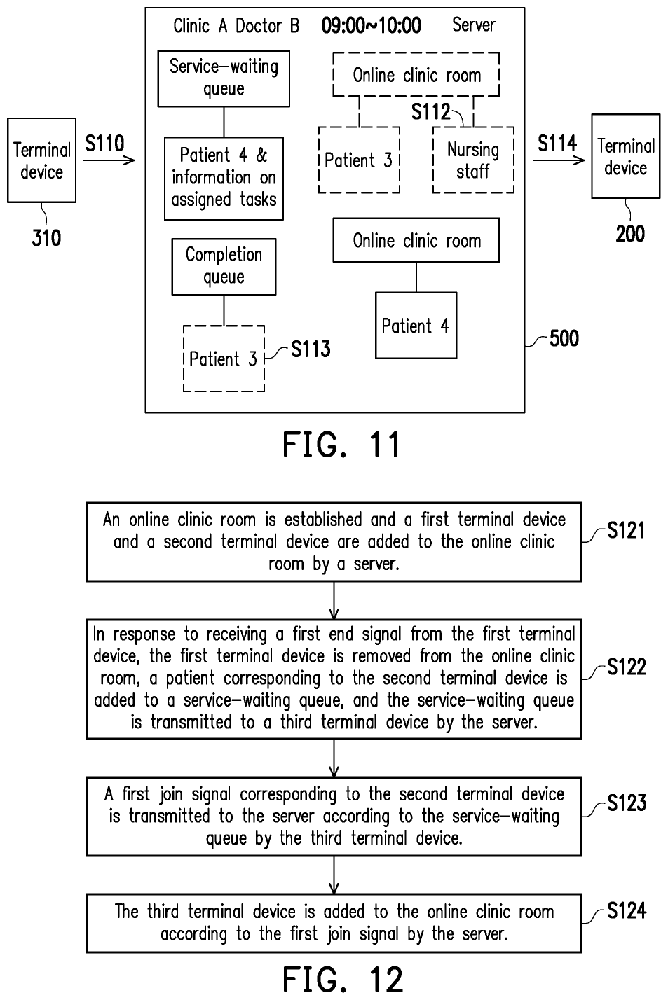

FIG. 11 is a schematic diagram illustrating a procedure for performing assigned tasks from a doctor according to still another embodiment of the disclosure. After a nursing staff member holding the terminal device 310 has completed assigned tasks for a patient, if all the assigned tasks related to the patient have been completed, the terminal device 200 may leave the online clinic room to end the entire diagnosis and treatment process.

In step S111, the terminal device 310 may transmit an end signal to the server 500.

In step S112, the server 500 may remove the terminal device 200 and the terminal device 310 from the online clinic room according to the end signal from the terminal device 310. To be specific, in response to the end signal not including information on assigned tasks, or in response to the information on assigned tasks carried by the end signal only including completed tasks but not pending tasks, the server 500 may decide to remove the terminal device 200 and the terminal device 310 from the online clinic room. In one embodiment, the server 500 may respectively remove the terminal device 310 and the terminal device 200 from the online clinic room in response to receiving the end signal from the terminal device 310 and the terminal device 200 respectively. After the terminal device 310 and the terminal device 200 are removed from the online clinic room, the server 500 may close the online clinic room.

In step S113, the server 500 may add the patient holding the terminal device 200 to a completion queue in response to the online clinic room being closed. The completion queue may include one or more patients who have completed the diagnosis and treatment process. The terminal device may access the completion queue stored in the server 500 to obtain the relevant information of the patients.

In step S114, the server 500 may transmit a notification signal to the terminal device 200 to remind the patient that the diagnosis and treatment process is completed.

FIG. 12 is a flowchart illustrating a remote diagnosis and treatment method according to an embodiment of the disclosure, wherein the remote diagnosis and treatment method may be implemented by the remote diagnosis and treatment system as shown in FIG. 1. In step S121, an online clinic room is established and a first terminal device and a second terminal device are added to the online clinic room by a server. In step S122, in response to receiving a first end signal from the first terminal device, the first terminal device is removed from the online clinic room, a patient corresponding to the second terminal device is added to a service-waiting queue, and the service-waiting queue is transmitted to a third terminal device by the server. In step 5123, a first join signal corresponding to the second terminal device is transmitted to the server according to the service-waiting queue by the third terminal device. In step S124, the third terminal device is added to the online clinic room according to the first join signal by the server.

In summary, the remote diagnosis and treatment system according to the disclosure enables the patient to complete online check-in procedure, and enables both the hospital and the patient to ensure that the patient is ready for diagnosis and treatment. The patient can judge the time to contact the hospital according to the treatment-waiting queue or the service-waiting queue provided by the remote diagnosis and treatment system, so as to prevent the patient from wasting a great deal of time waiting for the contact from the hospital. If the patient misses the number-calling, the remote diagnosis and treatment system can also remind the patient. In addition, the doctor may assign tasks to the nursing staff after the diagnosis and treatment is completed according to the needs of the patient. The nursing staff can know clearly through the remote diagnosis and treatment system which patient has completed the diagnosis and treatment and which patient needs further services from one or more nursing staff members. After one or more nursing staff members have completed all the assigned tasks, the remote diagnosis and treatment system can notify the hospital that the diagnosis and treatment service for the patient is completed.

It will be apparent to those skilled in the art that various modifications and variations can be made to the structure of the disclosure without departing from the scope or spirit of the disclosure. In view of the foregoing, it is intended that the disclosure cover modifications and variations of this disclosure provided they fall within the scope of the following claims and their equivalents.

What is claimed is:

1. A remote diagnosis and treatment system, comprising:
a server;
a first terminal device, communicatively connected to the server;
a second terminal device, communicatively connected to the server; and
a third terminal device, communicatively connected to the server, wherein
the server establishes an online clinic room and adds the first terminal device and the second terminal device to the online clinic room;
in response to receiving a first end signal from the first terminal device, the server removes the first terminal device from the online clinic room, adds a patient corresponding to the second terminal device to a service-waiting queue, and transmit the service-waiting queue to the third terminal device;
the third terminal device transmits a first join signal corresponding to the second terminal device to the server according to the service-waiting queue; and
the server adds the third terminal device to the online clinic room according to the first join signal.

2. The remote diagnosis and treatment system according to claim 1, wherein the first end signal comprises information on assigned tasks, wherein the server adds the patient to the service-waiting queue in response to receiving the information on assigned tasks.

3. The remote diagnosis and treatment system according to claim 1, wherein the server removes the patient from the service-waiting queue in response to receiving the first join signal.

4. The remote diagnosis and treatment system according to claim 3, wherein in response to receiving a second end signal comprising an updated information on assigned tasks from the third terminal device, the server removes the third terminal device from the online clinic room and adds the patient back to the service-waiting queue so as to generate an updated service-waiting queue.

5. The remote diagnosis and treatment system according to claim 3, further comprising:
a fourth terminal device, communicatively connected to the server, wherein
the server transmits the updated service-waiting queue to the fourth terminal device;
the fourth terminal device transmits a second join signal corresponding to the second terminal device to the server according to the updated service-waiting queue; and
the server adds the fourth terminal device to the online clinic room according to the second join signal.

6. The remote diagnosis and treatment system according to claim 2, wherein the server closes the online clinic room in response to receiving a second end signal from the second terminal device and receiving a third end signal from the third terminal device room.

7. The remote diagnosis and treatment system according to claim 2, wherein the server transmits a notification signal to the second terminal device in response to receiving the information on assigned tasks, wherein the notification signal comprises the service-waiting queue.

8. The remote diagnosis and treatment system according to claim 1, wherein the third terminal device receives a user operation so as to select the patient from a plurality of patients in the service-waiting queue, and transmits the first join signal corresponding to the patient to the server.

9. The remote diagnosis and treatment system according to claim 1, wherein
the first terminal device receives a treatment-waiting queue from the server, wherein the treatment-waiting queue comprises the patient;
the first terminal device transmits a number-calling signal corresponding to the patient to the server according to the treatment-waiting queue;
the server transmits a dialing signal to the second terminal device according to the number-calling signal; and
the server adds the second terminal device to the online clinic room in response to receiving a first reply signal corresponding to the dialing signal from the second terminal device.

10. The remote diagnosis and treatment system according to claim 9, wherein
the server removes the patient from the treatment-waiting queue in response to receiving the number-calling signal; and
the server adds the patient back to the treatment-waiting queue in response to receiving a second reply signal corresponding to the dialing signal from the second terminal device.

11. The remote diagnosis and treatment system according to claim 9, wherein
the server removes the patient from the treatment-waiting queue in response to receiving the number-calling signal;
the second terminal device transmits a third reply signal corresponding to the dialing signal to the server in response to not receiving a user operation after a preset period of time after receiving the dialing signal; and
the server adds the patient back to the treatment-waiting queue in response to receiving the third reply signal from the second terminal device.

12. The remote diagnosis and treatment system according to claim 9, wherein
the first terminal device receives a user operation so as to select the patient from a plurality of patients in the treatment-waiting queue, and transmits the number-calling signal corresponding to the patient to the server.

13. The remote diagnosis and treatment system according to claim 9, wherein the second terminal device outputs a prompt message in response to receiving the dialing signal.

14. The remote diagnosis and treatment system according to claim 1, wherein
the second terminal device receives appointment information from the server, wherein the appointment information comprises a check-in period; and
the server adds the patient corresponding to the second terminal device to a treatment-waiting queue in response to receiving a check-in signal from the second terminal device during the check-in period, and transmits the treatment-waiting queue to the second terminal device.

15. The remote diagnosis and treatment system according to claim 14, wherein
the server transmits a notification signal to the second terminal device in response to not receiving the check-in signal during the preset period.

16. A remote diagnosis and treatment method, comprising:
establishing an online clinic room and adding a first terminal device and a second terminal device to the online clinic room by a server;
in response to receiving a first end signal from the first terminal device, removing the first terminal device from the online clinic room, adding a patient corresponding to the second terminal device to a service-waiting queue, and transmitting the service-waiting queue to a third terminal device by the server;
transmitting a first join signal corresponding to the second terminal device to the server according to the service-waiting queue by the third terminal device; and
adding the third terminal device to the online clinic room according to the first join signal by the server.
